(19) United States
(12) Patent Application Publication
(10) Pub. No.: US 2024/0024493 A1
(43) Pub. Date: Jan. 25, 2024

(54) PURIFICATION METHODS FOR LARGE SCALE SYNTHESIS OF CUCURBIT[7]URIL-PEG CONJUGATES

(71) Applicant: Portal Insulin LLC, Alpine, CA (US)

(72) Inventor: Matthew J. Webber, Granger, IN (US)

(21) Appl. No.: 18/212,886

(22) Filed: Jun. 22, 2023

Related U.S. Application Data
(60) Provisional application No. 63/354,459, filed on Jun. 22, 2022.

(51) Int. Cl. A61K 47/60; A61K 47/69

(52) U.S. Cl. CPC A61K 47/60; A61K 47/6949; A61K 47/6933

(57) ABSTRACT

Methods of purification for the large scale synthesis of CB[7]-PEG may include at least one of diafiltration or tangential flow filtration, column chromatography, affinity “pull-down” techniques, or selective precipitation methods. In one embodiment, the method includes providing a reaction mixture containing synthesized CB[7]-PEG, providing a membrane selected to be below the nominal molecular weight of CB[7]-PEG, and removing small molecular weight contaminant species from the reaction mixture using the membrane. In embodiments, regardless of which purification method is used, a copper catalyst component of the “click” chemistry reaction mixture may be removed using a commercially available metal-chelating resin.

### CROSS-REFERENCE TO PRIOR APPLICATIONS:

The present application claims the benefit of U.S. Provisional Patent Application No. 63/354,459, filed on Jun. 22, 2022, entitled PURIFICATION METHODS FOR LARGE SCALE SYNTHESIS OF CUCURBIT[7]URIL-PEG CONJUGATES,” the entire disclosure of which is hereby incorporated herein by reference.

### TECHNICAL FIELD:

The present invention relates to methods for large scale synthesis of Cucurbit[7]uril-PEG (CB[7]-PEG) conjugates, and in particular, to methods of purification of the end product.

### BACKGROUND OF THE INVENTION:

Excipients, the inactive ingredients in drug formulations, perform a number of functions and can facilitate improved protein stability, solubility and absorption.

For example, formulations for insulin analogues may contain multiple excipients including tonicity agents, preservatives, and stabilizing agents, which are selected to enhance insulin stability. As described in published PCT application WO 2021/119607 A1, formulating monomeric insulin requires new excipients which do not promote the R₆ hexamer, but that still endow insulin with sufficient stability to prevent aggregation and denaturation over time. In this context it is noted that while covalent PEGylation has been successful as a strategy to stabilize insulin in formulation, the extended pharmacokinetics in vivo associated with PEGylation is not desirable for rapid acting insulins. Moreover, significant liver toxicity has been associated with covalent PEGylated insulin, as demonstrated in the unsuccessful clinical use of the PEGylated insulin Lispro (Ly2605541).

A better excipient is needed for rapid acting insulins.

### BRIEF DESCRIPTION OF THE DRAWING:

FIG. 1 illustrates an example synthesis and purification of CB[7]—PEG₅ₖ, according to various embodiments.

### SUMMARY OF THE INVENTION:

Methods of purification for the large scale synthesis of CB[7]-PEG may include at least one of diafiltration or tangential flow filtration, column chromatography, affinity “pull-down” techniques, or selective precipitation methods.

Regardless of which of the purification approaches are pursued, it is economical to remove the copper catalyst component of the “click” chemistry reaction mixture using a commercially available metal-chelating resin.

### DETAILED DESCRIPTION OF THE INVENTION:

Recent research has demonstrated non-covalent modification of proteins as a strategy to enhance their stability in formulation. In particular, conjugation of a polyethylene glycol (PEG) chain to a cucurbit[7]uril (CB[7]) macrocycle creates a tool for non-covalent

PEGylation using host—guest binding between the CB[7]-PEG excipient and residues on the insulin protein. CB[7]-PEG has strong binding affinities for terminal aromatic amino acids such the N-terminal phenylalanine found on insulin making it an ideal candidate for host-guest binding. The dynamic binding of CB[7]-PEG to insulin is promising as a zo strategy for stabilizing insulin in formulation.

Thus, CB[7]-PEG may be used to stabilize insulin at formulation concentrations, affording insulin/CB[7]-PEG complex with faster diffusion rate than the insulin hexamer. Moreover, the modest affinity and rapid exchange kinetics of non-covalent association for the insulin/CB[7]-PEG complex assure rapid complex dissociation upon dilution in the body, minimally impacting pharmacokinetics and avoiding the liver toxicity previously seen for covalent PEGylated insulin. As a result, synthesis of CB[7]-PEG on a commercial or large scale may be an important process in the production of stabilized insulin formulations.

The extension of using CB[7]-PEG to stabilize other classes of biopharmaceutical agents is also a promising indication. In what follows, aspects of the large scale synthesis of CB[7]-PEG are described, including, for example, methods of purification of the end product.

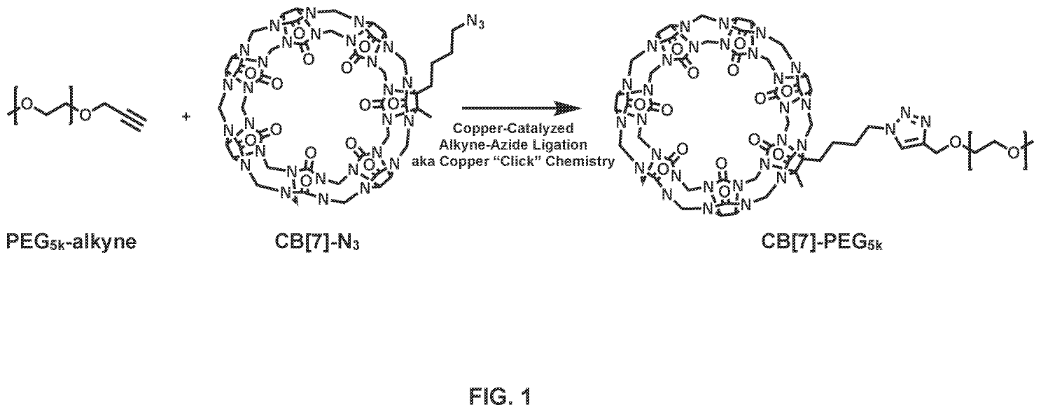

FIG. 1 illustrates a laboratory scale method of synthesis and purification of CB[7]-PEG₅ₖ. As noted in the figure, the example method shown in FIG. 1 utilizes copper-catalyzed “click chemistry” for the CB[7]-PEG synthesis. For the example process illustrated in FIG. 1, dialysis is the routine method of purification for this compound at laboratory scale.

Referring now to FIG. 1, in an example synthesis of CB[7]-PEG₅ₖ, 100 mg of CB[7]-N3 may be combined with 393 mg of PEG5k-Alkyne, copper(II) sulfate pentahydrate (CuSO4·5H20, 2.0 mg, BDH, ACS grade) and N,N,N′,N″,N″-pentamethyldiethylenetriamine (PMDETA, 98%, 1.6 μL), and dissolved in 8 mL DMF/water (1/1, v/v) in a Schlenk flask. The flask may be degassed with three freeze-pump-thaw cycles. On the last cycle, the flask may be opened to quickly add 20 mg of sodium ascorbate into the flask before re-capping the flask. The flask may then be vacuumed and backfilled with N2 over five (5) cycles before immersion in a 50° C. oil bath to thaw the solution and initiate the ‘click’ reaction. After 48 hours, the reaction may be quenched by exposure to air.

In an example purification process for this CB[7]-PEG synthesis, the reaction mixture may then be transferred into dialysis tubing (for example, MWCO=3500, Thermo Scientific) and dialyzed against 3 L water over 24 hours with a water change every 2 hours.

In example implementations of the above described synthesis and purification known to the inventor, the pure product was obtained after lyophilization as a yellow solid (0.49 g, 99% yield), and was determined by NMR to be fully substituted with CB[7].

However, the use of dialysis is not optimal for the production of CB[7]-PEG in a large scale industrial process. For example, the use of dialysis imposes a significant cost burden in terms of materials, and it is also not efficient in terms of time to purified product. Thus, in what follows, various alternatives are presented that may be used when purifying the CB[7]-PEG compound as part of a large scale synthesis.

Optional Pre-Purification & Copper Clean-Up:

Regardless of which of the below described large scale purification approaches are pursued, it is economically efficient to first remove the copper catalyst component of the “click” chemistry reaction mixture (shown in FIG. 1) using a commercially available metal-chelating resin. For example, the following products, or similar products, may be used as copper capture resins: Chelex 100, Dowex m4195, AmberSep TM IRC748 Chelating Resin, CupriSorb, Lewatit TP260, Dyna-Aqua™ Copper, and Alumina-based resins. In embodiments, industrial use of these resins is envisioned in the context of an affinity/ion exchange chromatography column, wherein the reaction cocktail (i.e., output reaction mixture) from the “click” chemistry ligation of CB[7] to PEG would be eluted over a bed of resin to capture and remove the copper catalyst contaminant.

In alternate embodiments, the resin may be used in a batch “pull-down” set-up, and use gravity or centrifuge to settle the resin and its bound copper, isolated away from the product supernatant phase. In embodiments, whichever resin is used, care must be taken that the CB[7]-PEG does not significantly adsorb to the resin and thus be unintentionally removed from the reaction mixture alongside the copper.

### 1. Diafiltration/Tangential Flow Filtration

In embodiments, the reaction mixture (with or without copper removal, as detailed above) may be purified using a flow-based alternative to dialysis, such as, for example, diafiltration or tangential flow filtration. By this method, the crude reaction mixture is purified using a commercially available, industrially scalable platform such as one of the following modular-based systems (or many similar systems being used in this area): Pellicon Ultrafiltration System, Pall Minimate Tangential Flow Filtration Capsules, Sartorius Vivaflow system, and Galen TFF-Easy system. Using this method, the crude reaction mixture may be cleaned up with the aid of a membrane (such as, for example, one having a 3 kDa molecular weight cut-off) that is selected to be below the nominal molecular weight of CB[7]-PEG (which is ˜6.2 kDa when prepared with a 5 kDa PEG chain) to enable purification of the compound through removal of the small molecular weight contaminant species (these include copper-binding ligand, sodium ascorbate, Dimethylformamide (“DMF”), etc.). In embodiments, this approach enables for a more rapid scale-up and purification, including solvent/buffer/salt exchange, if necessary, for clinical development.

As regards diafiltration/tangential flow filtration on CB[7]-PEG, challenges may arise from the dynamic, extended-chain conformation of the CB[7]-PEG relative to the more rigid/globular protein structures that these methods are typically employed to purify. More simply, the polymer may reptate/pass through pores much smaller than its nominal molecular weight, something a protein of similar size would be incapable of doing, and this issue may be exacerbated under flow conditions. Accordingly, in embodiments, care must be taken to mitigate against this challenge. If reptation of extended PEG coils across the diafiltration membrane is encountered, this issue may be mitigated by raising the temperature of the purification system from ambient (c.a. 22° C.) to elevated (c.a. ˜50° C.) to drive a coil-to-globule transition, thereby collapsing the PEG chains and reducing reputation of the extended coil form.

### 2. Column Chromatography Methods

In embodiments, the reaction mixture, with or without copper removal as described above, may be purified using some form of column chromatography. The following describes example column options and methodological formats for such methods.

A first example is reversed phase HPLC. Reverse-phase HPLC generally involves binding an organic molecule to a stationary phase, often silica derivatized with alkyl chains, in a relatively polar environment (the mobile phase), which could contain water, and then eluting the organic molecule using a gradient of a less polar organic solvent.

Thus, in embodiments, using a solid support presenting a saturated alkyl moiety (e.g., C₁₈), an example reaction mixture may be loaded to a column under aqueous conditions and then eluted by increasing the percent of a polar organic solvent, such as, for example, acetonitrile, ethanol or methanol. Although CB[7] may be expected to have some short-term retention due to interactions with alkyl chains on the column, it is noted that this approach may have some risk given the expectation for limited solubility of CB[7] (and to some extent PEG) in common organic solvents that would be used for the elution.

Another example is albumin chromatography. In embodiments, using a solid support presenting serum albumin, the reaction mixture may be loaded to a column under aqueous conditions and eluted with continued flow of the aqueous phase, optionally increasing salt and/or pH gradients. These columns are typically used in pharmaceutical purification to separate stereoisomers. Because CB[7] has some ability to bind proteins such as albumin with modest affinity (K_{eg}˜10⁵ M⁻¹), this type of chromatography may, in embodiments, be retrofitted to take advantage of this affinity. It is necessary, however, in such embodiments, to make sure that the density of albumin on the column is not problematic, if it enables high CB[7] retention.

Yet another example is Affinity Chromatography. CB[7] has wide-ranging affinity for a number of small molecule guests, such as, for example, aromatics, adamantanes, alkyl-amines, etc.. Thus, in some embodiments an affinity resin for CB[7] may be generated by presentation of a useful guest on a solid support. As an example, common guests zo including phenylalanine, diaminoethane, 4-(am inomethyl)benzoic acid, 1-adamantanecarboxylic acid, or related, may be covalently attached to certain amine- or carboxy-modified resins (such as, for example, silica or polystyrene porous microparticles) to prepare a solid support with affinity as a guest for the CB[7] module of CB[7]-PEG. In embodiments, the reaction mixture is loaded to such a column and eluted with pure water (optionally increasing salt and/or pH gradients), with retention of CB[7]-PEG to the affinity matrix, thereby enabling its purification from the reaction mixture. As with the exemplary albumin chromatography methods described above, it is noted that a high density of affinity groups on the resin may promote effectively irreversible CB[7] retention. Accordingly, in such embodiments, it is necessary to carefully choose resins where the CB[7]-PEG does not significantly adsorb to the resin or its modifications and unintentionally be removed from the mixture. This is because the binding behavior of CB[7] to certain solid surfaces can be surprising and difficult to predict, and may even prove effectively irreversible in its binding. Thus, in embodiments, careful selection of resin and useful guest needs to be performed. Significant retention of CB[7]-PEG may also be mitigated by eluting the affinity resin with a soluble guest, such as those proposed for resin modification in their soluble form, to isolate the CB[7]-PEG in complex with these guests. Example such guest chemistries, such as, for example, 3,3′-(Octane-1,8-diyl)bis(1-ehtyl-imidazolium) bromide, may be particularly useful as guests that may be subsequently removed from io the CB[7] portal through changes in pH, or introduction of the purified compound into organic solvents (e.g., methanol).

### 3. Affinity “Pull-Down” Methods

In some embodiments, the reaction mixture (with or without copper removal, as detailed above) may be processed using an affinity “pull-down” technique to isolate CB[7]-PEG from contaminants of the reaction mixture. By this method, resin bearing groups that bind CB[7] including albumin or small molecule guests—as detailed in the chromatography methods described above—may be used to isolate CB[7]-PEG in a batch “pull-down” setup using gravity or centrifuge to settle the resin and its bound CB[7]-PEG from the contaminant supernatant phase. Then, using, for example, dilution zo and/or a change in pH/osmolarity of the solution, the CB[7]-PEG may be released from the resin in its pure form.

### 4. Selective Precipitation Methods

In embodiments, the reaction mixture (with or without copper removal, as detailed above) may be processed to selectively precipitate and thereby isolate CB[7]-PEG from contaminants of the reaction mixture. It is noted that the “click” chemistry ligation is performed in a water/DMF mixture, under which conditions CB[7] and the final product are only somewhat soluble. Taking advantage of the very limited solubility of CB[7] in common organic solvents, such as, for example, methanol, acetone, dichloromethane, chloroform, and ethers, in embodiments, precipitation of the reaction mixture in a non- solvent for CB[7] may be performed. An ideal solvent would also be one for which the PEG component would have limited solubility. Accordingly, solvents that may be used include those used in PEG purification, such as, for example, cold alcohols (methanol, ethanol, isopropanol), ethers, ethylene glycol, hexane, and toluene. In embodiments, key considerations of this method include selection of a solvent which offers limited to no solubility of CB[7]-PEG, and yet readily solubilizes contaminants of the crude reaction mixture. For “click” chemistry, if copper is first removed using one of the affinity resins listed above, then, in embodiments, the remaining product and contaminants (such as copper-binding ligand, sodium ascorbate, and DMF) may then be processed io by addition to a bulk non-solvent for precipitation. The insoluble solid product may then, for example, be isolated by a process of centrifugation, decanting, and washing or by gravity/vacuum filtration with optional washing of the solid product or filter cake before drying.

It is noted in this context that, in embodiments, care should be taken to make sure that the precipitated solid is not difficult/impossible to resuspend. For example, in some circumstances the PEG chains could thread through the portals of CB[7] and form kinetically trapped solid precipitates that are not resolubilized in water, ruining future use of the compound. In such embodiments, this needs to be avoided. One route to mitigate this concern or resolubilize a kinetically trapped product is, for example, to zo adjust pH or osmolarity of the solubilizing solution to disfavor threading interactions, or, for example, to introduce a competing guest as a solubilizing agent, such as, for example, 3,3′-(Octane-1,8-diyl)bis(1-ehtyl-imidazolium) bromide, that could then be subsequently removed through changes in pH.

### Integration With Improved Methods Of Large Scale Synthesis

It is expected that future manifestations of the CB[7]-PEG synthesis technology will likely replace the “click” chemistry ligation with a more common conjugation approach. For example, CB[7] may be modified with an amine or a carboxylate, and then linked to a PEG chain bearing the complementary group (carboxylate or amine, respectively) through routine amide bond formation (e.g., carbodiimide coupling). In embodiments using such improved synthesis techniques, the need to remove copper and reagents from the standard “click” chemistry ligation is eliminated. Of course, any such alternate chemistries would still need a method for scalable purification. Importantly, the methods of the different approaches outlined above, as applied to synthesis of CB[7]-PEG prepared by “click” chemistry, would likewise be viable purification strategies for any such alternate chemistry.

Further, other methods, such as thiol-maleimide conjugation, for example, may be used to avoid purification entirely, though the stability of such compounds and possible reaction with thiols on insulin and related proteins are not fully known currently. Azide-DBCO conjugation, as reported in the initial work on these compounds, such as is described, for example, in U.S. Pat. No. 11,191,841 B2, also benefits from not requiring this extent of purification, may be used in some embodiments. It is noted that such Azide-DBCO conjugation may introduce economic issues related to cost of reagents as well as an increasingly hydrophobic conjugate that may limit high-concentration applications, and these issues would need to be managed in such embodiments.

In embodiments, the purification methods described hereinabove are significantly preferable to replicating lab-scale dialysis procedures at the manufacturing scale, due to significantly higher economic costs and lower scale of dialysis versus the alternatives zo noted here. The above described methods are not only applicable to large scale CB[7]-PEG synthesis, but are also transferrable to many envisioned future manifestations.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any zo specific value or sub-range within the stated ranges in different embodiments of the invention, to the tenth of the unit of the lower limit of the range, unless the context clearly dictates otherwise.

This application refers to various issued patents, published patent applications, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention may be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above description, but rather is as set forth in the appended claims. Those of ordinary skill in the art will appreciate that various changes and modifications to this description may be made without departing from the spirit or scope of the present invention, as defined in the following claims.

What is claimed:

1. A method of purification for a large scale synthesis of CB[7]-PEG, comprising:
providing a reaction mixture containing synthesized CB[7]-PEG; and
either:
using a solid support presenting a saturated alkyl moiety, loading the reaction mixture to a column under aqueous conditions and then eluting by increasing the percent of a polar organic solvent,
using a solid support presenting serum albumin, loading the reaction mixture to a column under aqueous conditions, and eluting it with continued flow of the aqueous phase, or
using a solid support presenting a useful guest for the CB[7] module of the CB[7]-PEG, loading the reaction mixture to a column under aqueous conditions, and eluting it with continued flow of the aqueous phase.

2. The method of claim 1, using a solid support presenting a saturated alkyl moiety, and wherein the polar organic solvent is at least one of acetonitrile, ethanol or methanol.

3. The method of claim 1, using a solid support presenting serum albumin, further comprising optionally increasing salt and/or pH gradients.

4. The method of claim 1, using a solid support presenting a useful guest for the CB[7] module of the CB[7]-PEG, wherein the useful guest is at least one of:
phenylalanine, diaminoethane, 4-(am inomethyl)benzoic acid, 1-adamantanecarboxylic acid, or a related molecule covalently attached to an amine- or carboxy-modified resin.

5. The method of claim 4, wherein the amine- or carboxy-modified resin is one of: silica or polystyrene porous microparticles.

6. The method of claim 5, further comprising eluting an affinity resin with the soluble guest to isolate the CB[7]-PEG in complex with the guest.

7. The method of claim 6, wherein the soluble guest is 3,3′-(Octane-1,8-diyl)bis(1-ehtyl-imidazolium) bromide.

8. The method of claim 6, wherein the soluble guest is a compound that is subsequently removed from the CB[7] portal through changes in pH or introduction of the purified compound into organic solvents.

9. The method of claim 1, further comprising first removing the copper catalyst component from the reaction mixture containing synthesized CB[7]-PEG using a commercially available metal-chelating resin.

10. The method of claim 9, wherein the commercially available metal-chelating resin is at least one of: Chelex 100, Dowex m4195, AmberSep™ IRC748 Chelating Resin, CupriSorb, Lewatit TP260, Dyna-Aqua™ Copper, and Alumina-based resins.

11. A method of purification for a large scale synthesis of CB[7]-PEG, comprising:
providing a reaction mixture containing synthesized CB[7]-PEG;
providing a membrane selected to be below the nominal molecular weight of CB[7]-PEG; and
removing small molecular weight contaminant species from the reaction mixture using the membrane.

12. The method of claim 11, wherein the small molecular weight contaminant species include at least one of:
copper-binding ligand, sodium ascorbate, and dimethylformamide (DMF).

13. The method of claim 11, further comprising raising the temperature of the purification system from ambient temperature to an elevated temperature of 50° C. or more to drive a coil-to-globule transition collapsing the PEG chains and reducing reputation of the extended coil form.

14-16. (canceled)

17. A method of purification for a large scale synthesis of CB[7]-PEG, comprising:
providing a reaction mixture containing synthesized CB[7]-PEG; and
either:
precipitating the reaction mixture in a non-solvent for CB[7] to selectively
precipitate CB[7]-PEG from contaminants of the reaction mixture;
or
providing a membrane selected to be below the nominal molecular weight of CB[7]-PEG; and
removing small molecular weight contaminant species from the reaction mixture using the membrane.

18. The method of claim 17, wherein the reaction mixture is precipitated in a non-solvent for CBI71, and wherein the non-solvent for CB[7] is at least one of: ethanol, isopropanol, methanol, acetone, dichloromethane, chloroform, or an ether.

19. The method of claim 17, wherein the reaction mixture is precipitated in a non-solvent for CB[7], and wherein the non-solvent for CB[7] has limited solubility of the PEG component.

20. The method of claim 19, wherein the non-solvent for CB[7] is at least one of: cold alcohols (methanol, ethanol, isopropanol), Ethers, Ethylene glycol, Hexane, or Toluene.

21. The method of claim 17, further comprising first removing the copper catalyst component from the reaction mixture containing synthesized CB[7]-PEG using a commercially available metal-chelating resin.

22. The method of claim 17, wherein the membrane selected to be below the nominal molecular weight of CB[7]-PEG is provided, and wherein the small molecular weight contaminant species include at least one of:
copper-binding ligand, sodium ascorbate, and dimethylformamide (DMF).

23. The method of claim 17, wherein the membrane selected to be below the nominal molecular weight of CB[7]-PEG is provided, and further comprising raising the temperature of the purification system from ambient temperature to an elevated temperature of 50° C. or more to drive a coil-to-globule transition collapsing the PEG chains and reducing reputation of the extended coil form.

24. The method of claim 11, further comprising first removing the copper catalyst component from the reaction mixture containing synthesized CB[7]-PEG using a commercially available metal-chelating resin.
